(19) United States
(12) Patent Application Publication
(10) Pub. No.: US 2021/0121358 A1
(43) Pub. Date: Apr. 29, 2021

(54) Method of Treating Peyronie's Disease

(71) Applicant: Don Velazquez, San Diego, CA (US)

(72) Inventor: Don Velazquez, San Diego, CA (US)

(21) Appl. No.: 17/080,770

(22) Filed: Oct. 26, 2020

Related U.S. Application Data
(60) Provisional application No. 62/926,402, filed on Oct. 25, 2019.

(51) Int. Cl. A61H 19/00

(52) U.S. Cl. CPC A61H 19/32; A61H 2203/0406; A61H 2201/163; A61H 2201/1621; A61H 2201/1616; A61H 2201/165; A61H 2201/1642; A61H 2203/0468; A61H 2201/1652; A61H 2201/1664; A61H 2201/1611

(57) ABSTRACT

An improved device and method for treating Peyronie's disease is disclosed. The improved device and method allows use of the device in pbulic.

### PRIORITY

This application claims priority to U.S. Provisional Application 62/926,402, filed 25 Oct. 2019.

### FIELD

The present invention is directed to an improved device and method for treating Peyronie's disease.

### BACKGROUND

Peyronie's disease is condition caused by scar tissue, called plaque, that forms inside the penis. It can result in a bent, rather than straight, erect penis. Treatment of the disease includes use of surgery, pills, topical cream, and/or medical devices applied to the penis. Medical devices for the treatment of Peyronie's disease include penile traction and vacuum erection devices. Penile traction devices typically comprise one or more clamping member, for example, a rubber ring or band, attached around the head of the penis; one or more base portion attached around the base of the penis; and one or more adjustable portion, for example, a rod and spring, is positioned along the penis and is connected at ends to the base and clamping portions. A force applied by the adjustable portion is used to cause the clamping member to move away from the base member, which causes the head of the penis to move sufficiently away from the base of the penis. Repeated use of the traction device is used to reduce or eliminate any bend or curvature in the penis. Known traction devices apply force to the distal end of the penis in a direction that extends the penis perpendicularly outward from the body. This outward extension combined with their rigid construction makes known penile traction devices unsuited for use in public. The outward extension and bulkiness of known traction devices can also limit the physical movement of patients, for example, sleeping in a prone position is made almost impossible when wearing known traction devices.

### FIGURES

FIG. 1 is a representation of a first portion of the present invention;

FIG. 2 is a representation of an orientation of a second and third portion of the present invention that is used to stretch a penis in a vertical upward direction;

FIG. 3 is another representation of an orientation of a second and third portion of the present invention that is used to stretch a penis in an angled upward direction;

FIG. 4 is another representation of an orientation of a second and third portion of the present invention that is used to stretch a penis in a vertical upward direction;

FIG. 5 is another representation of an orientation of a second and third portion of the present invention that is used to stretch a penis in an angled upward direction;

FIG. 6 is another representation of an orientation of a second and third portion of the present invention that is used to stretch a penis in an angled downward direction; and

FIG. 7 is another representation of an orientation of a second and third portion of the present invention that is used to stretch a penis in an angled downward direction.

### SUMMARY

The present invention is directed to treatment of Peyronie's disease in a manner that improves user comfort and enables use in public, as well as during sleep. With the present invention, during treatment of Peyronie's disease, a stretching force is applied to the penis in a direction that is generally along a patient's body, rather than in a direction that is perpendicular to the body.

In one embodiment, the present invention comprises a method of treating Peyronie's disease, comprising the steps of: attaching a first portion of a device to a penis of a patient; attaching a second portion of the device to a body portion of the patient that is not the penis; and applying a force on the penis with the device to cause the penis to straighten and thereby treat the Peyronie's disease. In one embodiment, the second portion is above the groin of the patient. In embodiments, the second portion is the waist, the chest, the shoulders, or the neck of the patient. In one embodiment, the second portion is below the groin of the person. In one embodiment, the second portion is the thigh, the knee, the calf, or the ankle of the patient. In one embodiment, the device applies the force with the patient in a prone position. In one embodiment, the patient in a standing position, the force is applied in an upward or downward direction. In one embodiment, the first portion is configured to secure the device to the penis via a clamping and/or compressive force. In one embodiment, the first portion comprises a ring. In one embodiment, the first portion applies the clamping and/or compressive force to a distal portion of the penis. In one embodiment, the first portion comprises a silicone, elastic, and/or rubber material. In one embodiment, the first portion encircles the head of the penis. In one embodiment, the first portion comprises an adhesive. In one embodiment, the second portion comprises a harness. In one embodiment, the second portion comprises one or more of a strap, tube, band, belt, and/or rope. In one embodiment, the second portion is configured to be attached to a thigh, knee, calf, ankle, neck, chest, and/or waist of the patient. In one embodiment, the first portion and the second portion are joined by a third portion. In one embodiment, the third portion is configured to apply the pulling force to penis. In one embodiment, the third portion comprises an elastic or stretchable material. In one embodiment, the third portion comprises one or more of a strap, tube, rope, belt, and/or band. In one embodiment, the force comprises a pulling or stretching force.

### DESCRIPTION

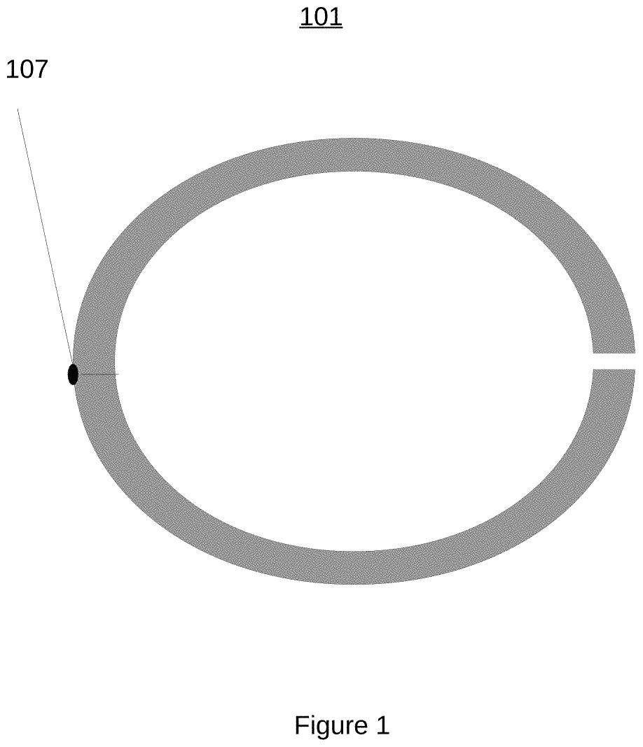

With reference to FIG. 1, in embodiments, the present invention utilizes a Peyronie's treatment device comprised of a respective first portion configured to be securable fastened to a penis of a patient being treated. In one embodiment, a first portion 101 comprises two half rings configured to be joined together over a penis. In one embodiment, the first portion comprises an inner opening that can be configured to securely fit over a penis via adjustment of a size of the opening. In one embodiment, the first portion comprises a fastening device, for example, a clasp or clamp. In one embodiment, the first portion is configured to be fastened to the head and/or shaft of a penis in a position that is distal from a base of the penis. In one embodiment, the first portion is configured to be fastened distal of patient's body and the curved or bent portion of the penis to be treated. In embodiments, the first portion comprises a silicone, a plastic and/or a rubber material. In embodiments, the first portion is covered or coated with silicone, elastic, and/or a rubber material. In one embodiment, the first portion is ring shaped. In one embodiment, the first portion is elastic and/or flexible. In one embodiment, the first portion comprises a rubber band or ring.

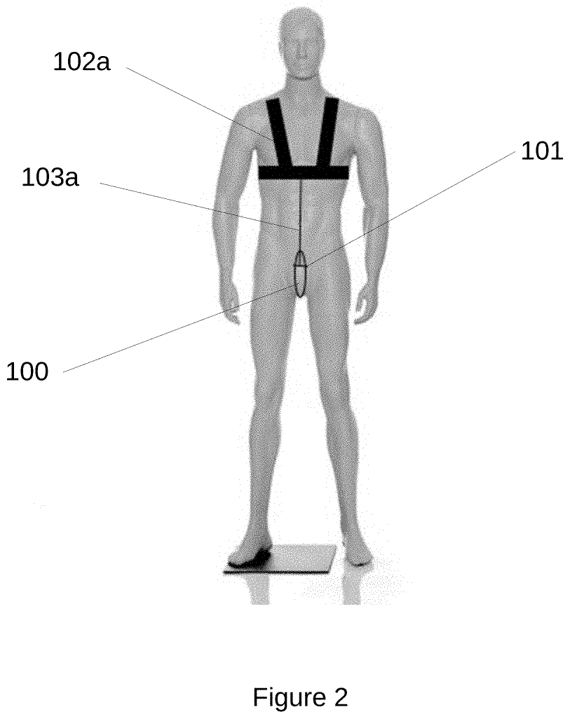

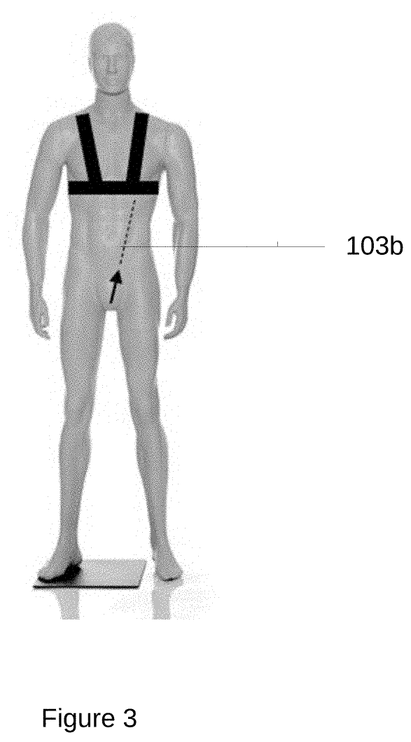

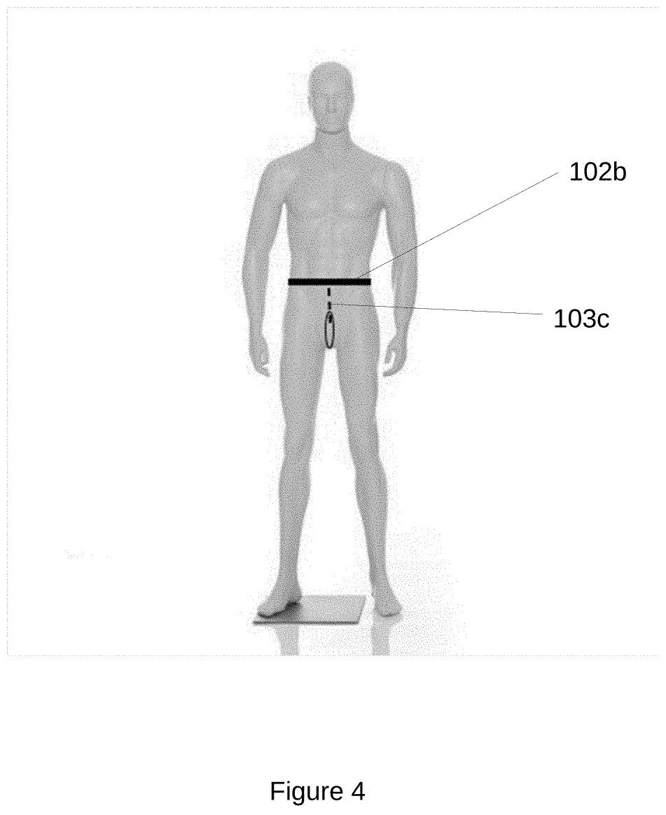

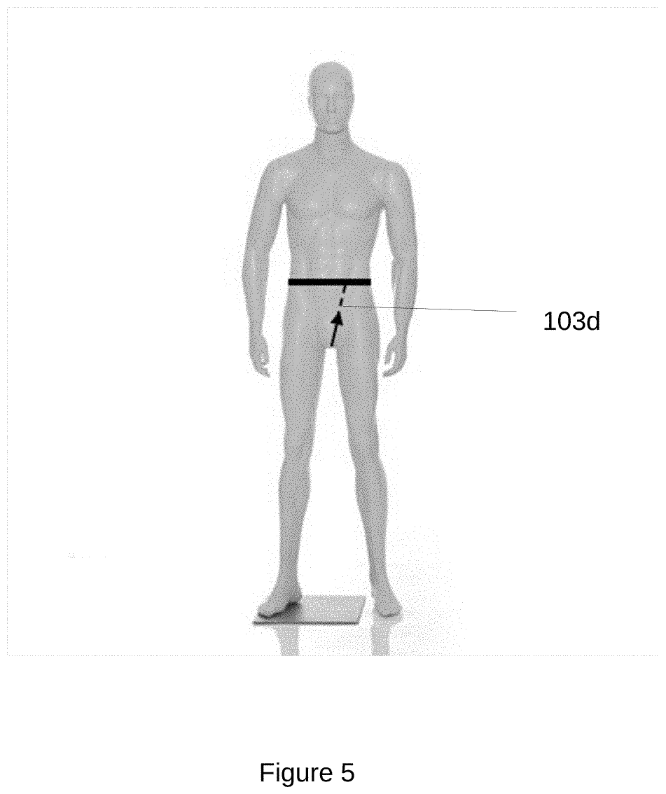

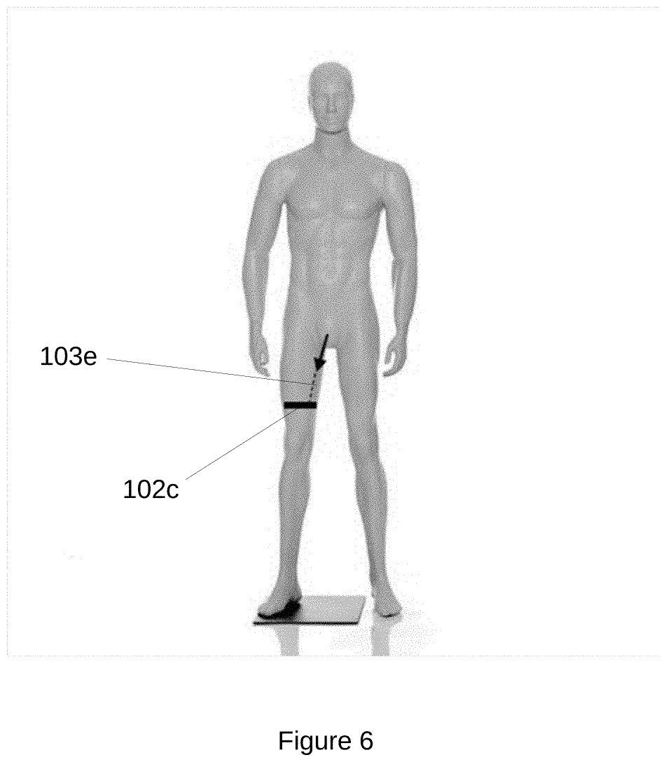

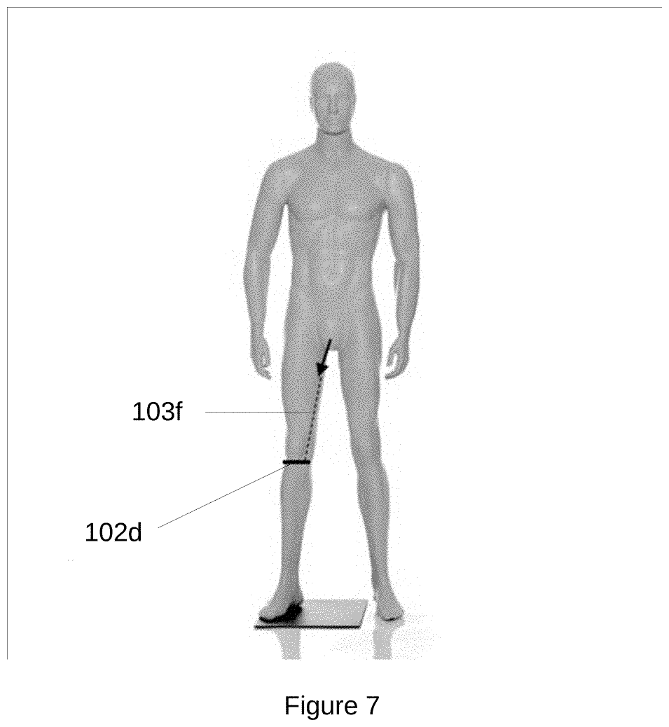

With reference to FIGS. 2-7, in embodiments, the present invention also utilizes a Peyronie's treatment device comprised of a second portion 102a or 102b. In one embodiment, a second portion comprises a harness configured to be worn over the body of a patient. In embodiments, one or more second portion comprised of one or more of a strap, tube, band, belt, and/or rope. In embodiments, one or more second portion comprises stretchable, flexible and/or elastic material known to those skilled in the body harness arts.

In one embodiment, second portion 102a is configured to be mounted over the chest, shoulders, and/or neck of a patient.

In one embodiment, second portion 102b is configured to be mounted over the waist of a patient.

In one embodiment, second portion 102c is configured to be mounted above either the right or left knee, for example, either thigh of a patient.

In one embodiment, second portion 102d is configured to be mounted below either the right or left knee, for example, either calf of the patient.

With reference to FIGS. 2-7, in embodiments, the present invention also utilizes a Peyronie's treatment device comprised of at least one respective third portion 103a, 103b, 103c, 103d, 103e, or 103f configured to apply a pulling force to a penis. In embodiments, an end of a third portion is attached to a second portion and an opposite end of a third portion is coupled to a first portion using techniques known to those skilled in the art. In embodiments, the location of and attachment point of a third portion to a second portion may be varied to achieve a desired pulling force and therapeutic effect. In one embodiment, a third portion is attached to a second portion along a sagittal plane of a patient to cause the first portion and, thus, the patient's penis to be pulled straight up along the patient's groin. In embodiments, a third portion is attached to a second portion offset from a sagittal plane of a patient to cause the first portion and, thus, the patient's penis to be pulled generally upward to the right or left along the patient's groin. In embodiments, a third portion is attached to a second portion offset from a sagittal plane of a patient to cause the first portion and, thus, the patient's penis to be pulled generally downward to the right or left along the patient's groin. In embodiments, one or more third portion comprises one or more of a strap, tube, rope, belt, and/or band. In embodiments, a third portion comprises a stretchable, flexible and/or elastic material. In embodiments, a position and/or length of the third portion is selected to achieve a desired therapeutic effect and/or to improve a patient's comfort.

What is claimed is:

1. A method of treating Peyronie's disease, comprising the steps of:
attaching a first portion of a device to a penis of a patient;
attaching a second portion of the device to a body portion of the patient that is not the penis; and
applying a force on the penis with the device to cause the penis to straighten and thereby treat the Peyronie's disease.

2. The method of claim 1, wherein with the patient in a standing position, the second portion is above the groin of the patient.

3. The method of claim 1, wherein the second portion is the waist, the chest, the shoulders, or the neck of the patient.

4. The method of claim 1, wherein with the patient in a standing position, the second portion is below the groin of the person.

5. The method of claim 1, wherein the second portion is the thigh, the knee, the calf, or the ankle of the patient.

6. The method of claim 1, wherein the device is configured to apply the force with the patient in a prone position.

7. The method of claim 1, wherein with the patient in a standing position, the force is applied in an upward or downward direction.

8. The method of claim 1, wherein the first portion is configured to secure the device to the penis via a clamping and/or compressive force.

9. The method of claim 8, wherein the first portion applies the clamping and/or compressive force to a distal portion of the penis.

10. The method of claim 9, wherein the first portion comprises a silicone, elastic, and/or rubber material.

11. The method of claim 1, wherein the first portion is configured to encircle the head of the penis.

12. The method of claim 1, wherein the first portion comprises an adhesive.

13. The method of claim 1, wherein the second portion comprises a harness.

14. The method of claim 1, wherein the second portion comprises one or more of a strap, tube, band, belt, and/or rope.

15. The method of claim 1, wherein the second portion is configured to be attached to a thigh, knee, calf, ankle, neck, chest, and/or waist of the patient.

16. The method of claim 1, wherein the first portion and the second portion are joined by a third portion.

17. The method of claim 16, wherein the third portion is configured to apply the pulling force to penis.

18. The method of claim 16, wherein the third portion comprises an elastic or stretchable material.

19. The method of claim 17, wherein the third portion comprises one or more of a strap, tube, rope, belt, and/or band.

20. The method of claim 1, wherein the force comprises a pulling or stretching force.
